(19) United States
(12) Patent Application Publication
(10) Pub. No.: US 2024/0215849 A1
(43) Pub. Date: Jul. 4, 2024

(54) INTRACRANIAL RADIO FREQUENCY COIL FOR INTRAOPERATIVE MAGNETIC RESONANCE IMAGING

(71) Applicant: Neuro42 Inc., San Francisco, CA (US)

(72) Inventors: Hung-Yu LIN, Walnut Creek, CA (US); Yong PANG, San Francisco, CA (US); Sree Poojitha YERRAMSETTI, San Francisco, CA (US)

(73) Assignee: Neuro42 Inc., San Francisco, CA (US)

(21) Appl. No.: 18/147,542

(22) Filed: Dec. 28, 2022

(51) Int. Cl. A61B 5/055; G01R 33/34

(52) U.S. Cl. CPC A61B 5/055; G01R 33/34007; G01R 33/3802

(57) ABSTRACT

The present disclosure provides systems and methods for magnetic resonance imaging using a radio frequency coil, such as, for example, an intracranial radio frequency coil. In at least one aspect, a system can include a surgical tool and a magnetic resonance imaging system. The surgical tool can include a distal end portion and a radio frequency reception coil attached to the distal end portion. The magnetic resonance imaging system can be configured to project a magnetic field within a field of view and intraoperatively image the radio frequency reception coil in the field of view.

### BACKGROUND

The present disclosure relates to magnetic resonance imaging (MRI), medical imaging, medical intervention, and surgical intervention. MRI systems often include large and complex machines that generate significantly high magnetic fields and create significant constraints on the feasibility of certain surgical interventions. Restrictions can include limited physical access to the patient by a surgeon and/or a surgical robot and/or limitations on the usage of certain electrical and mechanical components in the vicinity of the MRI scanner. Such limitations are inherent in the underlying design of many existing systems and are difficult to overcome.

### SUMMARY

In one aspect, the present disclosure describes a surgical system. The surgical system can include a surgical tool and a magnetic resonance imaging system. The surgical tool can include a distal end portion and a radio frequency reception coil attached to the distal end portion. The magnetic resonance imaging system can be configured to project a magnetic field within a field of view and intraoperatively image the radio frequency reception coil in the field of view.

In another aspect, the present disclosure describes a low-field magnetic resonance imaging system. The low-field magnetic resonance imaging system can include a dome-shaped housing, a surgical tool, and a control unit. The dome-shaped housing can define a region of interest. The dome-shaped housing can include an array of permanent magnets, a gradient coil assembly, and a radio frequency transmission coil. The array of permanent magnets can be configured to project a magnetic field into the region of interest. The radio frequency transmission coil can be configured to transmit radio frequency signals to excite magnetization in the region of interest. The surgical tool can include a radio frequency reception coil configured to receive radio frequency signals corresponding to the excited magnetization in the field of view. The control unit can include a processor and a memory communicatively coupled to said processor. The memory can store instructions executable by said processor to transmit radio frequency pulses to the radio frequency transmission coil and receive radio frequency signals from the radio frequency reception coil.

In yet another aspect, the present disclosure describes a method. The method can include positioning a housing around a patient's head. The housing can include a dome-shaped array of magnets configured to project a static magnetic field into a region of interest within the patient's head and a radio frequency transmission coil configured to excite magnetization in the region of interest. The method can further include inserting a surgical tool through an aperture in the housing into the region of interest. The surgical tool can include a radio frequency reception coil. The method can further include transmitting radio frequency pulses to the radio frequency transmission coil, receiving radio frequency signals from the radio frequency reception coil, and reconstructing an image of at least a portion of the patient's head from the radio frequency signals.

### BRIEF DESCRIPTION OF THE DRAWINGS

The various aspects described herein, both as to organization and methods of operation, together with further objects and advantages thereof, may best be understood by reference to the following description, taken in conjunction with the accompanying drawings as follows.

FIG. 1 depicts components of a MRI scanning system including af dome-shaped housing for a magnetic array, the dome-shaped housing surrounding a region of interest therein and further depicting the dome-shaped housing positioned to receive at least a portion of the head of a patient reclined on the table into the region of interest, in accordance with at least one aspect of the present disclosure.

FIG. 1A depicts a patient's head positioned in the region of interest of the MRI scanning system of FIG. 1.

FIG. 2 is a perspective view of an alternative dome-shaped housing for a magnetic array for use with the MRI scanning system of FIG. 1, wherein access apertures are defined in the dome-shaped housing, in accordance with at least one aspect of the present disclosure.

FIG. 3 is a perspective view of an alternative dome-shaped housing for a magnetic array for use with the MRI scanning system of FIG. 1, wherein access apertures and an adjustable gap is defined in the dome-shaped housing, in accordance with at least one aspect of the present disclosure.

FIG. 4 depicts a dome-shaped housing for use with a MRI scanning system having an access aperture in the form of a centrally-defined hole, in accordance with at least one aspect of the present disclosure.

FIG. 5 is a cross-sectional view of the dome-shaped housing of FIG. 4, in accordance with at least one aspect of the present disclosure.

FIG. 6 depicts a control schematic for a MRI system, in accordance with at least one aspect of the present disclosure.

FIG. 7 is a flowchart describing a method for obtaining imaging data from an MRI system, in accordance with at least one aspect of the present disclosure.

FIG. 8 depicts a MRI scanning system and a robotic system, in accordance with at least one aspect of the present disclosure.

FIG. 9 is illustrates RF reception coils and a Canadian penny, in accordance with various aspects of the present disclosure.

FIG. 10 is a schematic of an intracranial RF reception coil, in accordance with various aspects of the present disclosure.

FIG. 11 is a schematic of another intracranial RF reception coil, in accordance with various aspects of the present disclosure.

FIG. 12 is a schematic image illustrating a sensitive region of an example intracranial RF reception coil, in accordance with various aspects of the present disclosure.

FIG. 13 is a flowchart depicting a method of intraoperatively obtaining MR images of a patient's brain, in accordance with various aspects of the present disclosure.

Corresponding reference characters indicate corresponding parts throughout the several views. The exemplifications set out herein illustrate various disclosed embodiments, is one form, and such exemplifications are not to be construed as limiting the scope thereof in any manner.

### DETAILED DESCRIPTION

Applicant of the present application also owns the following patent applications that were filed on even date herewith and which are each incorporated by reference herein in their respective entireties:
U.S. Patent Application Attorney Docket No. 220407, titled MODULARIZED MULTI-PURPOSE MAGNETIC RESONANCE PHANTOM.
U.S. Patent Application Attorney Docket No. 220409, titled DEEP LEARNING SUPER-RESOLUTION TRAINING FOR ULTRA LOW-FIELD MAGNETIC RESONANCE IMAGING.

Applicant of the present application also owns the following patent applications, which are each incorporated by reference herein in their respective entireties:
International Patent Application No. PCT/US2022/72143, titled NEURAL INTERVENTIONAL MAGNETIC RESONANCE IMAGING APPARATUS, filed May 5, 2022.
U.S. patent application Ser. No. 18/057,207, titled SYSTEM AND METHOD FOR REMOVING ELECTROMAGNETIC INTERFERENCE FROM LOW-FIELD MAGNETIC RESONANCE IMAGES, filed Nov. 19, 2022.

Before explaining various aspects of interventional magnetic resonance imaging devices in detail, it should be noted that the illustrative examples are not limited in application or use to the details of construction and arrangement of parts illustrated in the accompanying drawings and description. The illustrative examples may be implemented or incorporated in other aspects, variations and modifications, and may be practiced or carried out in various ways. Further, unless otherwise indicated, the terms and expressions employed herein have been chosen for the purpose of describing the illustrative examples for the convenience of the reader and are not for the purpose of limitation thereof. Also, it will be appreciated that one or more of the following-described aspects, expressions of aspects, and/or examples, can be combined with any one or more of the other following-described aspects, expressions of aspects and/or examples.

Various aspects are directed to neural interventional magnetic resonance imaging (MRI) devices that allows for the integration of surgical intervention and guidance with an MRI. This includes granting physical access to the area around the patient as well as access to the patient's head with one or more access apertures. In addition, the neural interventional MRI device may allow for the usage of robotic guidance tools and/or traditional surgical implements. In various instances, a neural interventional MRI can be used intraoperatively to obtain scans of a patient's head and/or brain during a surgical intervention, such as a surgical procedure like a brain biopsy or neurosurgery.

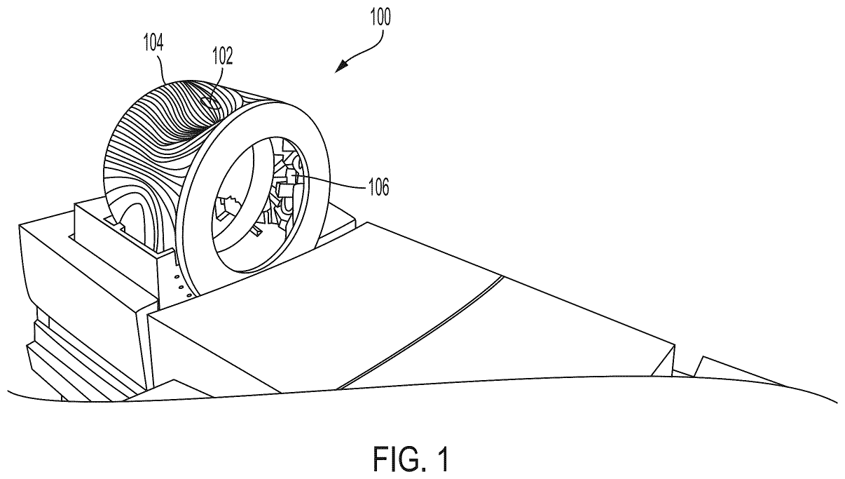

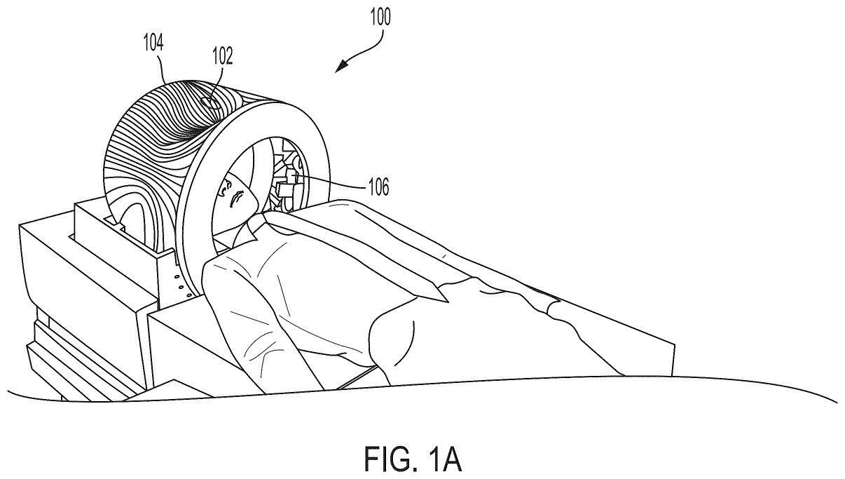

FIG. 1 depicts a MRI scanning system 100 that includes a dome-shaped housing 102 configured to receive a patient's head. The dome-shaped housing 102 can further include at least one access aperture configured to allow access to the patient's head to enable a neural intervention. A space within the dome-shaped housing 102 forms the region of interest for the MRI scanning system 100. Target tissue in the region of interest is subjected to magnetization fields/pulses, as further described herein, to obtain imaging data representative of the target tissue.

For example, referring to FIG. 1A, a patient can be positioned such that his/her head is positioned within the region of interest within the dome-shaped housing 102. The brain can be positioned entirely within the dome-shaped housing 102. In such instances, to facilitate intracranial interventions (e.g. neurosurgery) in concert with magnetic resonance (MR) imaging, the dome-shaped housing 102 can include one or more apertures that provide access to the brain. Apertures can be spaced apart around the perimeter of the dome-shaped housing.

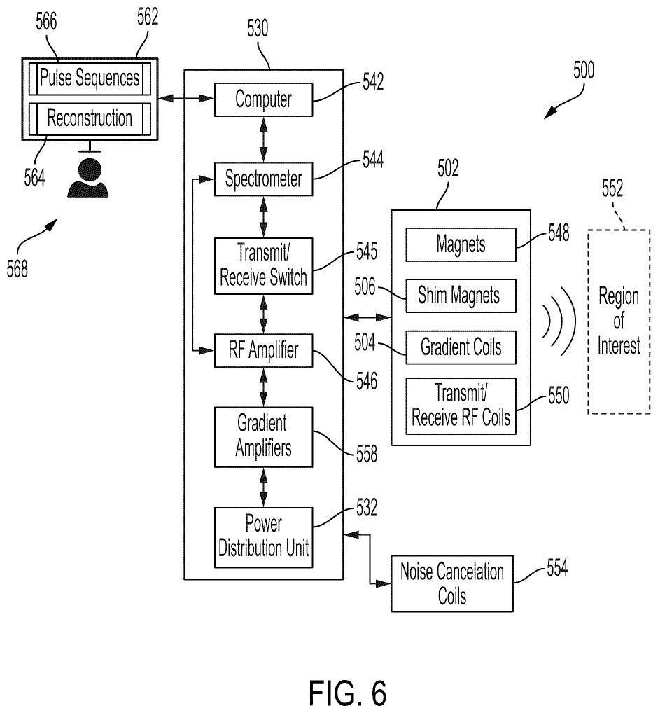

The MRI scanning system 100 can include an auxiliary cart (see, e.g. auxiliary cart 540 in FIG. 6) that houses certain conventional MRI electrical and electronic components, such as a computer, programmable logic controller, power distribution unit, and amplifiers, for example. The MRI scanning system 100 can also include a magnet cart that holds the dome-shaped housing 102, gradient coil(s), and/or a transmission coil, as further described herein. Additionally, the magnet cart can be attached to a receive coil in various instances. Referring primarily to FIG. 1, the dome-shaped housing 102 can further include radio frequency (RF) transmission coils, gradient coils 104 (depicted on the exterior thereof), and shim magnets 106 (depicted on the interior thereof). Alternative configurations for the gradient coil(s) 104 and/or shim magnets 106 are also contemplated. In various instances, the shim magnets 106 can be adjustably positioned in a shim tray within the dome-shaped housing 102, which can allow a technician to granularly configure the magnetic flux density of the dome-shaped housing 102.

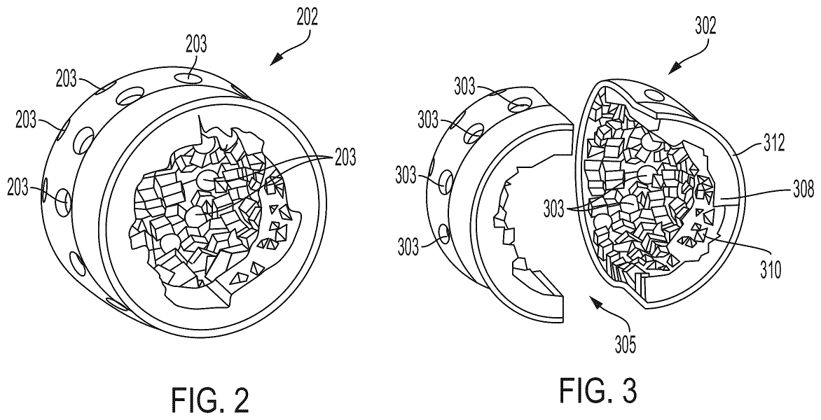

Various structural housings for receiving the patient's head and enabling neural interventions can be utilized with a MRI scanning system, such as the MRI scanning system 100. In one aspect, the MRI scanning system 100 may be outfitted with an alternative housing, such as a dome-shaped housing 202 (FIG. 2) or a two-part housing 302 (FIG. 3) configured to form a dome-shape. The dome-shaped housing 202 defines a plurality of access apertures 203; the two-part housing 302 also defines a plurality of access apertures 303 and further includes an adjustable gap 305 between the two parts of the housing.

In various instances, the housings 202 and 302 can include a bonding agent 308, such as an epoxy resin, for example, that holds a plurality of magnetic elements 310 in fixed positions. The plurality of magnetic elements 310 can be bonded to a structural housing 312, such as a plastic substrate, for example. In various aspects, the bonding agent 308 and structural housing 312 may be non-conductive or diamagnetic materials. Referring primarily to FIG. 3, the two-part housing 302 comprises two structural housings 312. In various aspect, a structural housing for receiving the patient's head can be formed from more than two sub-parts. The access apertures 303 in the structural housing 312 provide a passage directly to the patient's head and are not obstructed by the structural housing 312, bonding agent 308, or magnetic elements 310. The access apertures 303 can be positioned in an open space of the housing 302, for example.

There are many possible configurations of neural interventional MRI devices that can achieve improved access for surgical intervention. Many configurations build upon two main designs, commonly known as the Halbach cylinder and the Halbach dome described in the following article: Cooley et al. (e.g. Cooley, C. Z., Haskell, M. W., Cauley, S. F., Sappo, C., Lapierre, C. D., Ha, C. G., Stockmann, J. P., & Wald, L. L. (2018). Design of sparse Halbach magnet arrays for portable MRI using a genetic algorithm. IEEE transactions on magnetics, 54(1), 5100112. The article “Design of sparse Halbach magnet arrays for portable MRI using a genetic algorithm” by Cooley et al., published in IEEE transactions on magnetics, 54(1), 5100112 in 2018, is incorporated by reference herein in its entirety.

In various instances, a dome-shaped housing for an MRI scanning system, such as the system 100, for example, can include a Halbach dome defining a dome shape and configured based on several factors including main magnetic field B₀ strength, field size, field homogeneity, device size, device weight, and access to the patient for neural intervention. In various aspects, the Halbach dome comprises an exterior radius and interior radius at the base of the dome. The Halbach dome may comprise an elongated cylindrical portion that extends from the base of the dome. In one aspect, the elongated cylindrical portion comprises the same exterior radius and interior radius as the base of the dome and continues from the base of the dome at a predetermined length, at a constant radius. In another aspect, the elongated cylindrical portion comprises a different exterior radius and interior radius than the base of the dome (see e.g. FIGS. 2 and 3). In such instances, the different exterior radius and interior radius of the elongated cylindrical portion can merge with the base radii in a transitional region.

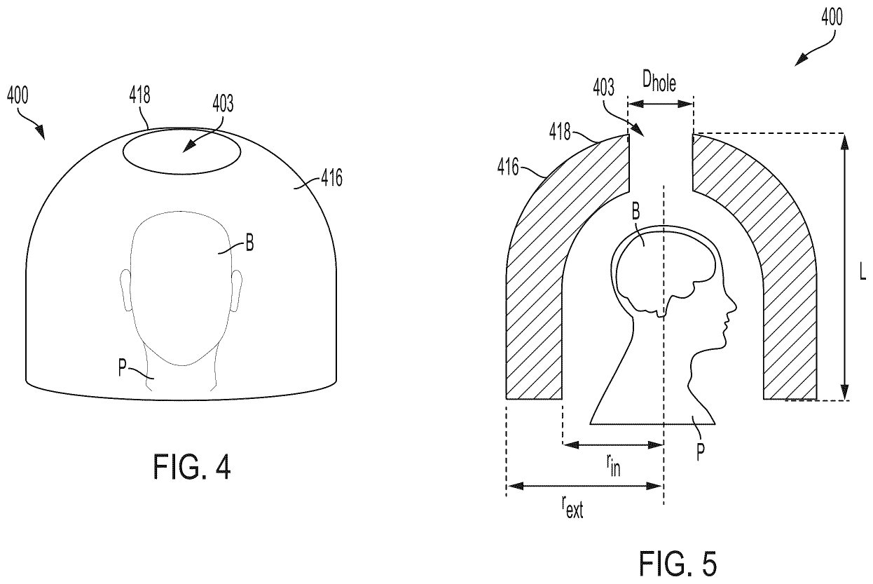

FIG. 4 illustrates an exemplary Halbach dome 400 for an MRI scanning system, such as the system 100, for example, which defines an access aperture in the form of a hole or access aperture 403, where the dome 400 is configured to receive a head and brain B of the patient P within the region of interest therein, and the access aperture 403 is configured to allow access to the patient P to enable neural intervention with a medical instrument and/or robotically-controlled surgical tool, in accordance with at least one aspect of the present disclosure. The Halbach dome 400 can be built with a single access aperture 403 at the top side 418 of the dome 400, which allows for access to the top of the skull while minimizing the impact to the magnetic field. Additionally or alternatively, the dome 300 can be configured with multiple access apertures around the structure 416 of the dome 400, as shown in FIGS. 2 and 3.

The diameter Dₕₒₗₑ of the access aperture 403 may be small (e.g. about 2.54 cm) or very large (substantially the exterior rₑₓₜ diameter of the dome 400). As the access aperture 403 becomes larger, the dome 400 begins to resemble a Halbach cylinder, for example. The access aperture 403 is not limited to being at the apex of the dome 400. The access aperture 403 can be placed anywhere on the surface or structure 416 of the dome 400. In various instances, the entire dome 400 can be rotated so that the access aperture 403 can be co-located with a desired physical location on the patient P.

FIG. 5 depicts relative dimensions of the Halbach dome 400, including a diameter Dₕₒₗₑ of the access aperture 403, a length L of the dome 400, and an exterior radius Text and an interior radius rᵢₙ of the dome 400. The Halbach dome 400 comprises a plurality of magnetic elements that are configured in a Halbach array and make up a magnetic assembly. The plurality of magnetic elements may be enclosed by the exterior radius rₑₓₜ and interior radius rᵢₙ in the structure 416 or housing thereof. In one aspect, example dimensions may be defined as: rᵢₙ=19.3 cm; rₑₓₜ=23.6 cm; L=38.7 cm; and 2.54 cm≤D<19.3 cm.

Based on the above example dimensions, a Halbach dome 400 with an access aperture 403 may be configured with a magnetic flux density B₀ of around 72 mT, and an overall mass of around 35 kg. It will be appreciated that the dimensions may be selected based on particular applications to achieve a desired magnetic flux density B₀, total weight of the Halbach dome 400 and/or magnet cart, and geometry of the neural intervention access aperture 403.

In various aspects, the Halbach dome 400 may be configured to define multiple access apertures 403 placed around the structure 416 of the dome 400. These multiple access apertures 403 may be configured to allow for access to the patient's head and brain B using tools (e.g., surgical tools) and/or a surgical robot.

In various aspects, the access aperture 403 may be adjustable. The adjustable configuration may provide the ability for the access aperture 403 to be adjusted using either a motor, mechanical assist, or a hand powered system with a mechanical iris configuration, for example, to adjust the diameter Dₕₒₗₑ of the access aperture 403. This would allow for configuration of the dome without an access aperture 403, conducting an imaging scan, and then adjusting the configuration of the dome 400 and mechanical iris thereof to include the access aperture 403 and, thus, to enable a surgical intervention therethrough.

Halbach domes and magnetic arrays thereof for facilitating neural interventions are further described in International Patent Application No. PCT/US2022/72143, titled NEURAL INTERVENTIONAL MAGNETIC RESONANCE IMAGING APPARATUS, filed May 5, 2022, which is incorporated by reference herein in its entirety.

Referring now to FIG. 6, a schematic for an MRI system 500 is shown. The MRI scanning system 100 (FIG. 1) and the various dome-shaped housings and magnetic arrays therefor, which are further described herein, for example, can be incorporated into the MRI system 500, for example. The MRI system 500 includes a housing 502, which can be similar in many aspects to the dome-shaped housings 102 (FIG. 1), 202 (FIG. 2), and/or 302 (FIG. 3), for example. The housing 502 is dome-shaped and configured to form a region of interest, or field of view, 552 therein. For example, the housing 502 can be configured to receive a patient's head in various aspects of the present disclosure.

The housing 502 includes a magnet assembly 548 having a plurality of magnets arranged therein (e.g. a Halbach array of magnets). In various aspect, the main magnetic field B₀, generated by the magnetic assembly 548, extends into the field of view 552, which contains an object (e.g. the head of a patient) that is being imaged by the MRI system 500.

The MRI system 500 also includes RF transmit/receive coils 550. The RF transmit/receive coils 550 are combined into integrated transmission-reception (Tx/Rx) coils. In other instances, the RF transmission coil can be separate from the RF reception coil. For example, the RF transmission coil(s) can be incorporated into the housing 502 and the RF reception coil(s) can be positioned within the housing 502 to obtain imaging data.

The housing 502 also includes one or more gradient coils 504, which are configured to generate gradient fields to facilitate imaging of the object in the field of view 552 generated by the magnet assembly 548, e.g., enclosed by the dome-shaped housing and dome-shaped array of magnetic elements therein. Shim trays adapted to receive shim magnets 506 can also be incorporated into the housing 502.

During the imaging process, the main magnetic field B₀ extends into the field of view 552. The direction of the effective magnetic field (B₁) changes in response to the RF pulses and associated electromagnetic fields transmitted by the RF transmit/receive coils 550. For example, the RF transmit/receive coils 550 may be configured to selectively transmit RF signals or pulses to an object in the field of view 552, e.g. tissue of a patient's brain. These RF pulses may alter the effective magnetic field experienced by the spins in the sample tissue.

The housing 502 is in signal communication with an auxiliary cart 530, which is configured to provide power to the housing 502 and send/receive control signals to/from the housing 502. The auxiliary cart 530 includes a power distribution unit 532, a computer 542, a spectrometer 544, a transmit/receive switch 545, an RF amplifier 546, and gradient amplifiers 558. In various instances, the housing 502 can be in signal communication with multiple auxiliary carts and each cart can support one or more of the power distribution unit 532, the computer 542, the spectrometer 544, the transmit/receive switch 545, the RF amplifier 546, and/or the gradient amplifiers 558.

The computer 542 is in signal communication with a spectrometer 544 and is configured to send and receive signals between the computer 542 and the spectrometer 544. When the object in the field of view 552 is excited with RF pulses from the RF transmit/receive coils 550, the precession of the object results in an induced electric current, or MR current, which is detected by the RF transmit/receive coils 550 and sent to the RF preamplifier 556. The RF preamplifier 556 is configured to boost or amplify the excitation data signals and send them to the spectrometer 544. The spectrometer 544 is configured to send the excitation data to the computer 542 for storage, analysis, and image construction. The computer 542 is configured to combine multiple stored excitation data signals to create an image, for example. In various instances, the computer 542 is in signal communication with at least one database 562 that stores reconstruction algorithms 564 and/or pulse sequences 566. The computer 542 is configured to utilize the reconstruction algorithms to generate an MR image 568.

From the spectrometer 544, signals can also be relayed to the RF transmit/receive coils 550 in the housing 502 via an RF power amplifier 546 and the transmit/receive switch 545 positioned between the spectrometer 544 and the RF power amplifier 546. From the spectrometer 544, signals can also be relayed to the gradient coils 560 in the housing 502 via a gradient power amplifier 558. For example, the RF power amplifier 546 is configured to amplify the signal and send it to RF transmission coils 560, and the gradient power amplifier 558 is configured to amplify the gradient coil signal and send it to the gradient coils 560.

In various instances, the MRI system 500 can include noise cancellation coils 554. For example, the auxiliary cart 530 and/or computer 542 can be in signal communication with noise cancellation coils 554. In other instances, the noise cancellation coils 554 can be optional. For example, certain MRI systems disclosed herein may not include supplemental/auxiliary RF coils for detecting and canceling electromagnetic interference, i.e. noise.

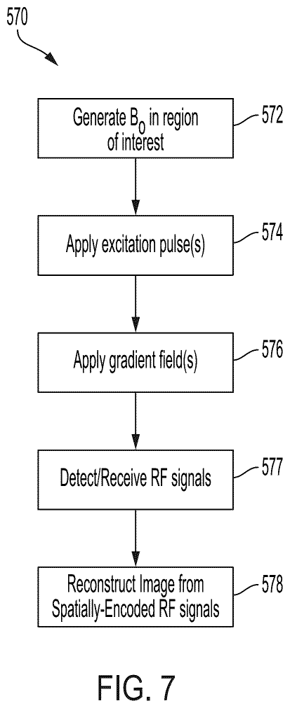

A flowchart depicting a process 570 for obtaining an MRI image is shown in FIG. 7. The flowchart can be implemented by the MRI system 500, for example. In various instances, at block 572, the target subject (e.g. a portion of a patient's anatomy), is positioned in a main magnetic field B₀ in an interest of region (e.g. region of interest 552), such as within the dome-shaped housing of the various MRI scanners further described herein (e.g. magnet assembly 548). The main magnetic field B₀ is configured to magnetically polarize the hydrogen protons (1H-protons) of the target subject (e.g. all organs and tissues) and is known as the net longitudinal magnetization M₀. It is proportional to the proton density (PD) of the tissue and develops exponentially in time with a time constant known as the longitudinal relaxation time T1 of the tissue. T1 values of individual tissues depend on a number of factors including their microscopic structure, on the water and/or lipid content, and the strength of the polarizing magnetic field, for example. For these reasons, the T1 value of a given tissue sample is dependent on age and state of health.

At block 574, a time varying oscillatory magnetic field B₁, i.e. an excitation pulse, is applied to the magnetically polarized target subject with a RF coil (e.g. RF transmit/receive coil 550). The carrier frequency of the pulsed B₁ field is set to the resonance frequency of the 1H-proton, which causes the longitudinal magnetization to flip away from its equilibrium longitudinal direction resulting in a rotated magnetization vector, which in general can have transverse as well as longitudinal magnetization components, depending on the flip angle used. Common B₁ pulses include an inversion pulse, or a 180-degree pulse, and a 90-degree pulse. A 180-degree pulse reverses the direction of the 1H-proton's magnetization in the longitudinal axis. A 90-degree pulse rotates the 1H-proton's magnetization by 90 degrees so that the magnetization is in the transverse plane. The MR signals are proportional to the transverse components of the magnetization and are time varying electrical currents that are detected with suitable RF coils. These MR signals decay exponentially in time with a time constant known as the transverse relaxation time T2, which is also dependent on the microscopic tissue structure, water/lipid content, and the strength of the magnetic field used, for example.

At block 576, the MR signals are spatially encoded by exposing the target subject to additional magnetic fields generated by gradient coils (e.g. gradient coils 560), which are known as the gradient fields. The gradient fields, which vary linearly in space, are applied for short periods of time in pulsed form and with spatial variations in each direction. The net result is the generation of a plurality of spatially encoded MR signals, which are detected at block 577, and which can be reconstructed to form MR images depicting slices of the examination subject. A RF reception coil (e.g. RF transmit/receive coil 550) can be configured to detect the spatially-encoded RF signals. Slices may be oriented in the transverse, sagittal, coronal, or any oblique plane.

At block 578, the spatially encoded signals of each slice of the scanned region are digitized and spatially decoded mathematically with a computer reconstruction program (e.g. by computer 542) in order to generate images depicting the internal anatomy of the examination subject. In various instances, the reconstruction program can utilize an (inverse) Fourier transform to back-transforms the spatially-encoded data (k-space data) into geometrically decoded data.

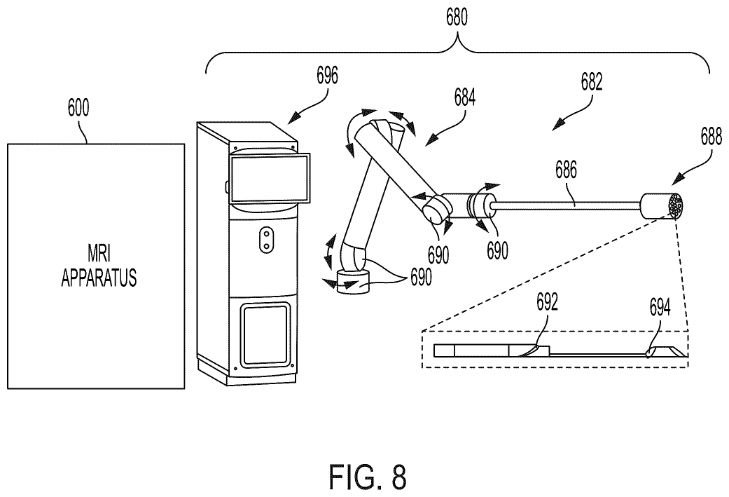

FIG. 8 depicts a graphical illustration of a robotic system 680 that may be used for neural intervention with an MRI scanning system 600. The robotic system 680 includes a computer system 696 and a surgical robot 682. The MRI scanning system 600 can be similar to the MRI system 500 and can include the dome-shaped housing and magnetic arrays having access apertures, as further described herein. For example, the MRI system 500 can include one or more access apertures defined in a Halbach array of magnets in the permanent magnet assembly to provide access to one or more anatomical parts of a patient being imaged during a medical procedure. In various instances, a robotic arm and/or tool of the surgical robot 682 is configured to extend through an access aperture in the permanent magnet assembly to reach a patient or target site. Each access aperture can provide access to the patient and/or surgical site. For example, in instances of multiple access apertures, the multiple access apertures can allow access from different directions and/or proximal locations.

In accordance with various embodiments, the robotic system 680 is configured to be placed outside the MRI system 600. As shown in FIG. 8, the robotic system 680 can include a robotic arm 684 that is configured for movements with one or more degrees of freedom. In accordance with various embodiments, the robotic arm 684 includes one or more mechanical arm portions, including a hollow shaft 686 and an end effector 688. The hollow shaft 686 and end effector 688 are configured to be moved, rotated, and/or swiveled through various ranges of motion via one or more motion controllers 690. The double-headed curved arrows in FIG. 8 signify exemplary rotational motions produced by the motion controllers 690 at the various joints in the robotic arm 684.

In accordance with various embodiments, the robotic arm 684 of the robotic system 682 is configured for accessing various anatomical parts of interest through or around the MRI scanning system 600. In accordance with various embodiments, the access aperture is designed to account for the size of the robotic arm 684. For example, the access aperture defines a circumference that is configured to accommodate the robotic arm 684, the hollow shaft 686, and the end effector 688 therethrough. In various instances, the robotic arm 684 is configured for accessing various anatomical parts of the patient from around a side of the magnetic imaging apparatus 600. The hollow shaft 686 and/or end effector 688 can be adapted to receive a robotic tool 692, such as a biopsy needle having a cutting edge 694 for collecting a biopsy sample from a patient, for example.

The reader will appreciate that the robotic system 682 can be used in combination with various dome-shaped and/or cylindrical magnetic housings further described herein. Moreover, the robotic system 682 and robotic tool 692 in FIG. 8 are exemplary. Alternative robotic systems can be utilized in connection with the various MRI systems disclosed herein. Moreover, handheld surgical instruments and/or additional imaging devices (e.g. an endoscope) and/or systems can also be utilized in connection with the various MRI systems disclosed herein.

In various aspects of the present disclosure, the MRI systems described herein can comprise low field MRI (LF-MRI) systems. In such instances, the main magnetic field B₀ generated by the permanent magnet assembly can be between 0.1 T and 1.0 T, for example. In other instances, the MRI systems described herein can comprise ultra-low field MRI (ULF-MRI) systems. In such instances, the main magnetic field B₀ generated by the permanent magnet assembly can be between 0.03 T and 0.1 T, for example.

Higher magnetic fields, such as magnetic fields above 1.0 T, for example, can preclude the use of certain electrical and mechanical components in the vicinity of the MRI scanner. For example, the existence of surgical instruments and/or surgical robot components comprising metal, specially ferrous metals, can be dangerous in the vicinity of higher magnetic fields because such tools can be drawn toward the source of magnetization. Moreover, higher magnetic fields often require specifically-designed rooms with additional precautions and shielding to limit magnetic interference. Despite the limitations on high field MRI systems, low field and ultra-low field MRI systems present various challenges to the acquisition of high quality images with sufficient resolution for achieving the desired imaging objectives.

LF- and ULF-MRI systems generally define an overall magnetic field homogeneity that is relatively poor in comparison to higher field MRI systems. For example, a dome-shaped housing for an array of magnets, as further described herein, can comprise a Halbach array of permanent magnets, which generate a magnetic field B₀ having a homogeneity between 1,000 ppm and 10,000 ppm in the region of interest in various aspects of the present disclosure.

In certain instances, an MRI system, such as the MRI scanning system 100 shown in FIG. 1, for example, can be utilized in concert with a neurosurgical intervention. For example, as shown in FIG. 1A, a patient can be positioned on the table and the patient's head can be positioned within the region of interest defined by the dome-shaped housing 102. A clinician can access the patient's brain with surgical devices via apertures in the dome-shaped housing 102. Moreover, the MRI scanning system 100 can be a LF-MRI or ULF-MRI. In various instances, surgical devices (e.g. handheld instruments and/or robotic tools) can be utilized in the presence of a low or ultra-low magnetic field, such that the MRI scanning system 100 can be utilized intraoperatively to image the patient's brain (or a portion thereof). Imaging may be obtained intraoperatively during a neurological procedure, such as a brain biopsy, for example.

In various instances, it can be challenging to obtain high-resolution MR images of a patient's brain (or portions thereof) especially with LF- or ULF-MRIs. Lower magnetic field strengths generally correspond to lower signal-to-noise ratios (SNRs) and correspondingly lower resolution. Additionally or alternatively, it can be challenging to intraoperatively identify and/or track the location of certain surgical devices in various instances, such as during a neurological procedure in which the patient's head is enclosed within an MRI system and/or at least partially surrounded by magnets and/or coils.

In various instances, an RF coil can be attached to a surgical device that is positioned in the patient's brain, such as a biopsy need, scalpel, or other surgical device that is positioned in the patient's brain. In some instances, the RF coil can be integrated into and/or supported by (e.g., wrapped about and/or extending through) the surgical device. In other instances, the RF coil can be integrated into and/or supported by (e.g., wrapped about and/or extending through) a separate substrate that is mounted to or otherwise attached to the surgical device. The RF coil can be an RF reception coil that is used in connection with an MRI system. In various instances, the placement of the RF reception coil within the region of interest (e.g. positioned within the patient's head and/or brain) can be configured to generate higher resolution MR images than those obtained with an external RF reception coil (e.g. positioned outside the patient's head and/or brain, such as within the housing of the MRI scanning system). Additionally or alternatively, the RF reception coil can be used to identify and/or track the position of the RF reception coil, and thus the surgical device to which it is attached, during a surgical intervention.

In various instances, an RF reception coil for an MRI system can be positioned within the field of view to increase the image resolution and/or facilitate tracking of a surgical device within the field of view. The RF reception coils can be much smaller than external MR reception coils. The compact size of such an RF reception coil enables insertion of the coil into the patient's anatomy (e.g. into the patient's brain). In various instances, such RF reception coils can be referred to as a “microcoils” owing to their relatively diminutive size in comparison to conventional external RF reception coils.

In certain instances, the RF reception coil can define a loop of wire having a diameter of less than 2.0 mm. In certain instances, the RF reception coil can define a loop of wire having a diameter of less than 2.0 mm and more than 1.0 mm. For example, in various instances, the RF reception coil can define a loop of wire having a diameter of 1.2 mm. Alternative coil diameters are contemplated. The reader will appreciate that the coil's diameter can be minimized to correspond to the diameter of a surgical device inserted into the patient's anatomy. Additionally or alternatively, the coil's diameter can be selected to avoid interference with anatomical structures otherwise unaffected by the surgical procedure or surgical intervention.

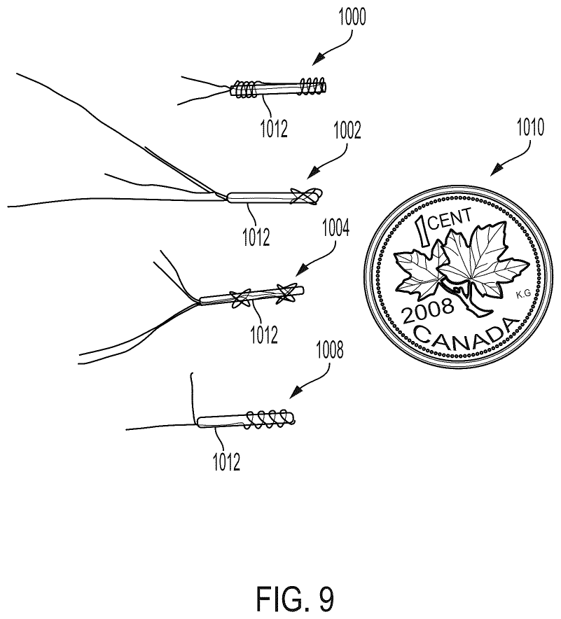

Referring now to FIG. 9, exemplary RF reception coils 1000, 1002, 1004, 1006 are shown relative to a Canadian penny 1010 for scale. For example, the Canadian penny 1010 has a diameter of 19.05 mm. Any of the RF reception coils 1000, 1002, 1004, 1006 can be integrated into and/or supported by (e.g., wrapped about and/or extending through) a substrate 1012. In some instances, the substrate 1012 can be a portion of a surgical device. In other instances, the substrate 1012 can be mounted to or otherwise supported by a surgical device. The exemplary RF reception coils 1000, 1002, 1004, 1006 and/or the substrate 1012 shown in FIG. 9 can be structured for use in neurosurgical procedures. For example, the RF reception coils 1000, 1002, 1004, 1006 and/or the substrate 1012 can be dimensioned for insertion into an intracranial vascular structure. In some instances, any of the RF reception coils 1000, 1002, 1004, 1006 can define a loop of wire having a diameter of less than 2.0 mm, such as a loop of wire having a diameter of less than 2.0 mm and more than 1.0 mm.

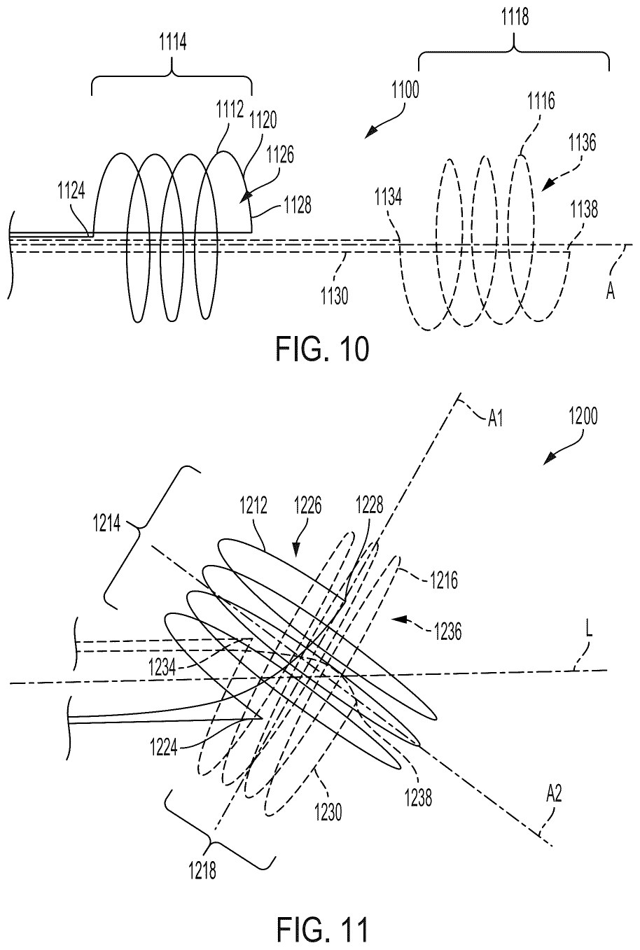

Referring now to FIGS. 10 and 11, RF reception coils 1100 (FIG. 10) and 1200 (FIG. 11) are shown. The RF reception coils 1100, 1200 can be used in connection with the MRI scanning system 100 (FIG. 1) and/or the MRI system 500 (FIG. 6). For example, referring primarily to FIG. 6, the RF reception coil 1100 or 1200 can be used along with the RF transmit/receive coils 550 in the MRI system 500 in various aspects of the present disclosure. For example, the RF transmit/receive coils 550 can include a first (or primary) receive coil, and the RF reception coil 1100 or 1200 can be a second (or secondary) receive coil for the MRI system 500. For example, the primary receive coil (e.g., RF transmit/receive coils 550) of the MRI system can be used to determine an intracranial position of surgical device having a secondary receive coil (e.g., the RF reception coil 1100 or 1200) attached thereto. Further, because the secondary receive coil can be positioned proximate to an intracranial region of interest, the secondary receive coil can be used to acquire high-resolution images the intracranial region of interest. Other aspects, the MRI system 500 can include separate transmit and receive coils. For example, a transmit-only coil can be incorporated into the housing 502, and the RF reception coil 1100 or 1200 can function as a receive-only coil that is configured to detect the magnetization induced by the RF pulses transmitted by the transmit-only coil.

Referring primarily to FIG. 10, the RF reception coil 1100 forms a double opposed solenoid having a first solenoid 1112 at a proximal portion 1114 of the RF reception coil 1100 and a second solenoid 1116 at a distal portion 118 of the RF reception coil 1100. The first solenoid 1112 is formed from a length of wire 1120 forming a helical coil having a proximal end 1124 and distal end 1128. At the distal end 1128 of the first solenoid 1112, the wire 1120 is routed back through a central aperture 1126 formed by the helical coil toward the proximal end 1124 of the first solenoid 1112. Lengths of the wire 1120 proximal to the proximal end 1124 of the first solenoid 1112 are substantially parallel and/or can merge toward parallel alignment.

Similarly, the second solenoid 1116 is formed from a length of wire 1130 forming a helical coil having a proximal end 1134 and a distal end 1138. At the distal end 1138 of the second solenoid 1116, the wire 1130 is routed back through a central aperture 1136 formed by the helical coil toward the proximal end 1134 of the second solenoid 1116. The wire 1130 is further routed back through the central aperture 1136 formed by the helical coil of the first solenoid 1112 toward the proximal end 1124 thereof. Lengths of the wire 1130 proximal to the proximal end 1134 of the second solenoid 1116 are substantially parallel and/or can merge toward parallel alignment.

The second solenoid 1116 is a longitudinally-offset mirror image reflection of the first solenoid 1112 about a longitudinal axis A of the RF reception coil 1100. In various instances, a double opposed solenoid, such as the RF reception coil 1100, for example, can be used for intracranial vascular vessel wall imaging. For example, the RF reception coil 1100 can be integrated into and and/or supported by surgical instrument, such as a biopsy needed or catheter, that is inserted into an intracranial vessel. The Intracranial vascular vessel wall imaging is further described herein.

Referring primarily to FIG. 11, the RF reception coil 1200 forms a double orthogonal solenoid having a first solenoid 1212 and a second solenoid 1216 that are rotationally offset and longitudinally overlap. The first solenoid 1212 is formed from a length of wire 1220 forming a helical coil having a proximal end 1224 and distal end 1228 and defining a central aperture 1226 that extends along an axis A1. At the distal end 1228 of the first solenoid 1212, the wire 1220 is routed back through the central aperture 1226 toward the proximal end 1224 of the first solenoid 1212. Proximal to the proximal end 1224 of the first solenoid 1212, the lengths of the wire 1220 merge toward substantially parallel alignment.

Similarly, the second solenoid 1216 is formed from a length of wire 1230 forming a helical coil having a proximal end 1234 and a distal end 1238. At the distal end 1238 of the second solenoid 1216, the wire 1230 is routed back through a central aperture 1236 formed by the helical coil toward the proximal end 1234 of the second solenoid 1216. Proximal to the proximal end 1234 of the second solenoid 1216, the lengths of the wire 1230 merge toward substantially parallel alignment.

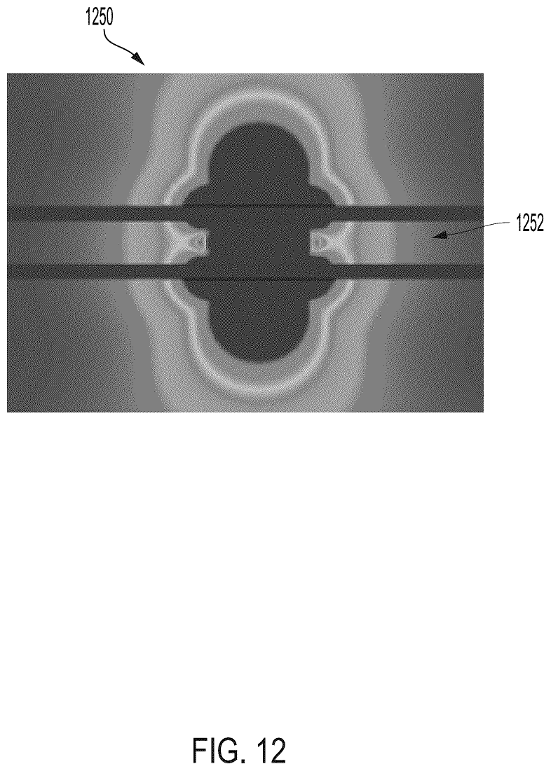

The second solenoid 1216 is a rotationally-offset from the first solenoid 1212 by 90 degrees. For example, axis A1 can be rotationally offset from the longitudinal axis L by 45 degrees in a first rotational direction, and axis A2 can be rotationally offset from the longitudinal axis L by 45 degrees in the opposite rotational direction. In various instances, the rotational offset between the first solenoid 1212 and the second solenoid 1216 can be less than 135 degrees and more than 45 degrees. The rotational offset can be selected to optimize imaging in a particular direction. For example, by rotating each solenoid symmetrically in opposite direction about a longitudinal axis L, such as 45 degrees, for example, the RF reception coil 1200 can be designed to support intracranial vascular surgical procedures. In some instances, the double orthogonal solenoid design of the RF reception coil 1200 can be configured to generate a sensitive region that enables imaging distally to the RF reception coil 1200 as the RF reception 1200 is advanced by a needle or catheter inside a cranial vascular system. By imaging distally to the RF reception coil 1200, a surgeon performing an intracranial vascular intervention using the RF reception coil 1200 can identify and/or avoid the penetration of critical cranial vascular structures. FIG. 12 is a schematic image illustrating an example sensitive region 1250 that may be associated with the RF reception coil 1200 of FIG. 11. As shown in FIG. 12, the sensitive region 1252 can include a distal region 1252 that extends distally from the RF reception coil. A method of intracranial vascular intervention is further described herein.

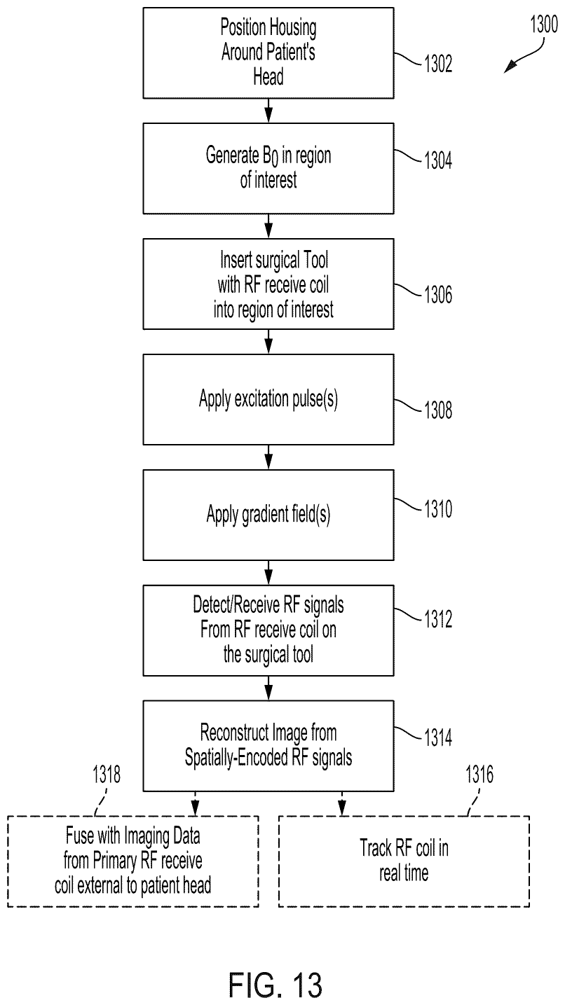

Referring now to FIG. 13, an imaging method 1300 is described. The imaging method 1300 can be used to image a patient's brain or a portion of a patient's brain. At block 1302, a MRI housing is positioned around a patient's head. For example, the dome-shaped housing 102 (FIG. 1), 202 (FIG. 2), or 302 (FIG. 3) can be positioned around a patient's head such that the region of interest is defined within the housing. The patient can be reclined on a table or sitting upright, for example.

At block 1304, a static magnetic field B₀ is generated and projected into the region of interest. The static magnetic field B₀ can be a low magnetic field or an ultra-low magnetic field. For example, the static magnetic field B₀ can be less than 1.0 T and, in various instances, can be 0.5 T, for example. In other instances, the static magnetic field B₀ can have a field strength higher than that of a low magnetic field or an ultra-low magnetic field.

A surgical tool having an RF reception coil is inserted into the region of interest at block 1306. The surgical tool can have a distal end comprising the RF reception coil, for example. The surgical tool can be inserted into the region of interest through an aperture in the housing in various instances. The surgical tool can be a biopsy needle or surgical scalpel, for example. Alternative surgical tools are also contemplated, such as, for example, probes, retractors, and dissectors.

At block 1308, excitation pulse(s) are transmitted to the region of interest and gradient field(s) are applied at block 1310. At block 1312, RF signals are detected by the RF reception coil mounted to the surgical tool in the region of interest. Blocks 1308, 1310, and 1312 can be implemented in accordance with a pulse sequence stored in the memory accessible to the computer/control circuit of the MRI system, for example. In various instances, the excitation pulses, gradient fields, and signal reception periods can be performed in a different order and/or at different repetition patterns according to the pulse sequence. At block 1314, an image of the patient's brain (or at least a portion of the patient's brain) can be generated from the spatially-encoded RF signals received from the RF receive coil according to the pulse sequence. In various instances, the local intracranial RF receive coil can enable ultra-high resolution images, for various applications, such as tumor characterization, for example.

In certain instances, as shown at block 1316, the RF coil can be tracked in real time. As a result, the clinician can track the position of the surgical tool onto which the RF receive coil is mounted in real time and intraoperatively.

Additionally or alternatively, in various instances, the reconstructed image from the spatially-encoded RF signals received by the RF receive coil can be fused with imaging data from another RF receive coil, such as a primary RF receive coil positioned external to the patient's brain, as shown at block 1318. In various instances, the primary RF receive coil can be positioned in the housing, for example. Image fusion from both a local viewpoint (via the intracranial RF receive coil) and a global viewpoint (via the external RF receive coil) can provide improved visualization for neurosurgeons in certain instances.

### EXAMPLES

Various additional aspects of the subject matter described herein are set out in the following numbered examples:

Example 1: A surgical system, comprising: a surgical tool comprising a distal end portion and a radio frequency reception coil attached to the distal end portion; and a magnetic resonance imaging system configured to project a magnetic field within a field of view, wherein the magnetic resonance imaging system is configured to intraoperatively image the radio frequency reception coil in the field of view.

Example 2: The surgical system of Example 1, wherein the surgical tool comprises a biopsy needle.

Example 3: The surgical system of Example 1, wherein the surgical tool comprises a scalpel.

Example 4: The surgical system of any one of Examples 1-3, wherein the radio frequency reception coil defines a loop of coil.

Example 5: The surgical system of any one of Examples 1-3, wherein the radio frequency reception coil comprises an outer diameter of less than 2 mm.

Example 6: The surgical system of Example 5, wherein the outer diameter is more than 1 mm.

Example 7: The surgical system of Example 6, wherein the outer diameter is 1.2 mm.

Example 8: The surgical system of any one of Examples 1-7, wherein the magnetic resonance imaging system is configured to track the radio frequency reception coil in real time.

Example 9: The surgical system of any one of Examples 1-8, wherein the magnetic resonance imaging system further comprises a primary radio frequency transmission coil configured to transmit radio frequency signals configured to excite magnetization in the field of view.

Example 10: The surgical system of Example 9, wherein the magnetic resonance imaging system further comprises a primary radio frequency reception coil configured to receive radio frequency signals corresponding to the excited magnetization in the field of view.

Example 11: A low-field magnetic resonance imaging system, comprising: a dome-shaped housing defining a region of interest, wherein the dome-shaped housing comprises: an array of permanent magnets configured to project a magnetic field into the region of interest; a gradient coil assembly; and a radio frequency transmission coil configured to transmit radio frequency signals to excite magnetization in the region of interest; a surgical tool comprising a radio frequency reception coil configured to receive radio frequency signals corresponding to the excited magnetization in the field of view; a control unit comprising a processor and a memory communicatively coupled to said processor, wherein said memory stores instructions executable by said processor to: transmit radio frequency pulses to the radio frequency transmission coil; and receive radio frequency signals from the radio frequency reception coil.

Example 12: The low-field magnetic resonance imaging system of Example 11, wherein the memory further stores instructions executable by the processor to reconstruct an image of the region of interest.

Example 13: The low-field magnetic resonance imaging system of any one of Examples 11 and 12, wherein the memory further stores instructions executable by the processor to track the radio frequency reception coil in the region of interest in real time.

Example 14: The low-field magnetic resonance imaging system of any one of Examples 11 and 12, wherein the magnetic field comprises a field strength of less than 1 T.

Example 15: The low-field magnetic resonance imaging system of Example 14, wherein the field strength comprises a field strength of less than 0.3 T.

Example 16: The low-field magnetic resonance imaging system of any one of Examples 11 and 12, wherein the surgical tool is selected from a group consisting of a biopsy needle and a scalpel.

Example 17: The low-field magnetic resonance imaging system of any one of Examples 11-16, wherein the radio frequency receive coil defines a loop of coil defining an outer diameter of less than 2 mm and more than 1 mm.

Example 18: A method, comprising: positioning a housing around a patient's head, wherein the housing comprises: a dome-shaped array of magnets configured to project a static magnetic field into a region of interest within the patient's head; and a radio frequency transmission coil configured to excite magnetization in the region of interest; inserting a surgical tool through an aperture in the housing into the region of interest, wherein the surgical tool comprises a radio frequency reception coil; transmitting radio frequency pulses to the radio frequency transmission coil; receiving radio frequency signals from the radio frequency reception coil; and reconstructing an image of at least a portion of the patient's head from the radio frequency signals.

Example 19: The method of Example 18, wherein the static magnetic field comprises a field strength of less than 1 T.

Example 20: The method of any one of Examples 18 and 19, further comprising tracking the radio frequency reception coil in real time.

Though various aspects disclosed herein are directed to brain imaging and/or neurological interventions, the reader will appreciate that the various systems and methods disclosed herein can be used to image other portions of a patient's anatomy and/or different structures in various instances.

While several forms have been illustrated and described, it is not the intention of Applicant to restrict or limit the scope of the appended claims to such detail. Numerous modifications, variations, changes, substitutions, combinations, and equivalents to those forms may be implemented and will occur to those skilled in the art without departing from the scope of the present disclosure. Moreover, the structure of each element associated with the described forms can be alternatively described as a means for providing the function performed by the element. Also, where materials are disclosed for certain components, other materials may be used. It is therefore to be understood that the foregoing description and the appended claims are intended to cover all such modifications, combinations, and variations as falling within the scope of the disclosed forms. The appended claims are intended to cover all such modifications, variations, changes, substitutions, modifications, and equivalents.

The foregoing detailed description has set forth various forms of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, and/or examples can be implemented, individually and/or collectively, by a wide range of hardware, software, firmware, or virtually any combination thereof. Those skilled in the art will recognize that some aspects of the forms disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as one or more program products in a variety of forms, and that an illustrative form of the subject matter described herein applies regardless of the particular type of signal bearing medium used to actually carry out the distribution.

Instructions used to program logic to perform various disclosed aspects can be stored within a memory in the system, such as dynamic random access memory (DRAM), cache, flash memory, or other storage. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, compact disc, read-only memory (CD-ROMs), and magneto-optical disks, read-only memory (ROMs), random access memory (RAM), erasable programmable read-only memory (EPROM), electrically erasable programmable read-only memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the non-transitory computer-readable medium includes any type of tangible machine-readable medium suitable for storing or transmitting electronic instructions or information in a form readable by a machine (e.g., a computer).

As used in any aspect herein, the term “control circuit” may refer to, for example, hardwired circuitry, programmable circuitry (e.g., a computer processor including one or more individual instruction processing cores, processing unit, processor, microcontroller, microcontroller unit, controller, digital signal processor (DSP), programmable logic device (PLD), programmable logic array (PLA), or field programmable gate array (FPGA)), state machine circuitry, firmware that stores instructions executed by programmable circuitry, and any combination thereof. The control circuit may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc. Accordingly, as used herein “control circuit” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

As used in any aspect herein, the term “logic” may refer to an app, software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

As used in any aspect herein, the terms “component,” “system,” “module” and the like can refer to a control circuit computer-related entity, either hardware, a combination of hardware and software, software, or software in execution.

As used in any aspect herein, an “algorithm” refers to a self-consistent sequence of steps leading to a desired result, where a “step” refers to a manipulation of physical quantities and/or logic states which may, though need not necessarily, take the form of electrical or magnetic signals capable of being stored, transferred, combined, compared, and otherwise manipulated. It is common usage to refer to these signals as bits, values, elements, symbols, characters, terms, numbers, or the like. These and similar terms may be associated with the appropriate physical quantities and are merely convenient labels applied to these quantities and/or states.

A network may include a packet switched network. The communication devices may be capable of communicating with each other using a selected packet switched network communications protocol. One example communications protocol may include an Ethernet communications protocol which may be capable permitting communication using a Transmission Control Protocol/Internet Protocol (TCP/IP). The Ethernet protocol may comply or be compatible with the Ethernet standard published by the Institute of Electrical and Electronics Engineers (IEEE) titled “IEEE 802.3 Standard”, published in December, 2008 and/or later versions of this standard. Alternatively or additionally, the communication devices may be capable of communicating with each other using an X.25 communications protocol. The X.25 communications protocol may comply or be compatible with a standard promulgated by the International Telecommunication Union-Telecommunication Standardization Sector (ITU-T). Alternatively or additionally, the communication devices may be capable of communicating with each other using a frame relay communications protocol. The frame relay communications protocol may comply or be compatible with a standard promulgated by Consultative Committee for International Telegraph and Telephone (CCITT) and/or the American National Standards Institute (ANSI). Alternatively or additionally, the transceivers may be capable of communicating with each other using an Asynchronous Transfer Mode (ATM) communications protocol. The ATM communications protocol may comply or be compatible with an ATM standard published by the ATM Forum titled “ATM-MPLS Network Interworking 2.0” published August 2001, and/or later versions of this standard. Of course, different and/or after-developed connection-oriented network communication protocols are equally contemplated herein.

Unless specifically stated otherwise as apparent from the foregoing disclosure, it is appreciated that, throughout the foregoing disclosure, discussions using terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

One or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

The terms “proximal” and “distal” are used herein with reference to a clinician manipulating the handle portion of the surgical instrument. The term “proximal” refers to the portion closest to the clinician and the term “distal” refers to the portion located away from the clinician. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flow diagrams are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures or characteristics may be combined in any suitable manner in one or more aspects.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

What is claimed is:

1. A surgical system, comprising:
a surgical tool comprising a distal end portion and a radio frequency reception coil attached to the distal end portion; and
a magnetic resonance imaging system configured to project a magnetic field within a field of view, wherein the magnetic resonance imaging system is configured to intraoperatively image the radio frequency reception coil in the field of view.

2. The surgical system of claim 1, wherein the surgical tool comprises a biopsy needle.

3. The surgical system of claim 1, wherein the surgical tool comprises a scalpel.

4. The surgical system of claim 1, wherein the radio frequency reception coil defines a loop of coil.

5. The surgical system of claim 1, wherein the radio frequency reception coil comprises an outer diameter of less than 2 mm.

6. The surgical system of claim 5, wherein the outer diameter is more than 1 mm.

7. The surgical system of claim 6, wherein the outer diameter is 1.2 mm.

8. The surgical system of claim 1, wherein the magnetic resonance imaging system is configured to track the radio frequency reception coil in real time.

9. The surgical system of claim 1, wherein the magnetic resonance imaging system further comprises a primary radio frequency transmission coil configured to transmit radio frequency signals configured to excite magnetization in the field of view.

10. The surgical system of claim 9, wherein the magnetic resonance imaging system further comprises a primary radio frequency reception coil configured to receive radio frequency signals corresponding to the excited magnetization in the field of view.

11. A low-field magnetic resonance imaging system, comprising:
a dome-shaped housing defining a region of interest, wherein the dome-shaped housing comprises:
an array of permanent magnets configured to project a magnetic field into the region of interest;
a gradient coil assembly; and
a radio frequency transmission coil configured to transmit radio frequency signals to excite magnetization in the region of interest;
a surgical tool comprising a radio frequency reception coil configured to receive radio frequency signals corresponding to the excited magnetization in the field of view;
a control unit comprising a processor and a memory communicatively coupled to said processor, wherein said memory stores instructions executable by said processor to:
transmit radio frequency pulses to the radio frequency transmission coil; and
receive radio frequency signals from the radio frequency reception coil.

12. The low-field magnetic resonance imaging system of claim 11, wherein the memory further stores instructions executable by the processor to reconstruct an image of the region of interest.

13. The low-field magnetic resonance imaging system of claim 11, wherein the memory further stores instructions executable by the processor to track the radio frequency reception coil in the region of interest in real time.

14. The low-field magnetic resonance imaging system of claim 11, wherein the magnetic field comprises a field strength of less than 1 T.

15. The low-field magnetic resonance imaging system of claim 14, wherein the field strength comprises a field strength of less than 0.3 T.

16. The low-field magnetic resonance imaging system of claim 11, wherein the surgical tool is selected from a group consisting of a biopsy needle and a scalpel.

17. The low-field magnetic resonance imaging system of claim 11, wherein the radio frequency receive coil defines a loop of coil defining an outer diameter of less than 2 mm and more than 1 mm.

18. A method, comprising:
positioning a housing around a patient's head, wherein the housing comprises:
a dome-shaped array of magnets configured to project a static magnetic field into a region of interest within the patient's head; and
a radio frequency transmission coil configured to excite magnetization in the region of interest;
inserting a surgical tool through an aperture in the housing into the region of interest, wherein the surgical tool comprises a radio frequency reception coil;
transmitting radio frequency pulses to the radio frequency transmission coil;
receiving radio frequency signals from the radio frequency reception coil; and
reconstructing an image of at least a portion of the patient's head from the radio frequency signals.

19. The method of claim 18, wherein the static magnetic field comprises a field strength of less than 1 T.

20. The method of claim 18, further comprising tracking the radio frequency reception coil in real time.
